(19) United States
(12) Patent Application Publication
(10) Pub. No.: US 2005/0080353 A1
(43) Pub. Date: Apr. 14, 2005

(54) Electronic device for measuring body fat, method of measuring body fat using the same, and health management system using the device and method

(76) Inventors: Jin-sang Whang, Suwon-si (KR); Hyung-sok Yeo, Suwon-si (KR); Kyung-ho Kim, Yongin-si (KR); Jeong-hwan Lee, Suwon-si (KR); Jae-chan Park, Daejeon-si (KR)

Correspondence Address: Eugene M. Lee;LEE & STERBA, PC, Suite 2000, 1101 Wilson Boulevard, Arlington, VA, 22209, US

(21) Appl. No.: 10/959,182

(22) Filed: Oct. 7, 2004

(30) Foreign Application Priority Data
Oct. 7, 2003 (KR) 2003-69703

(51) Int. Cl. A61B 5/05

(52) U.S. Cl. 600/547; 128/921

(57) ABSTRACT

An electronic device for measuring body fat, a method of measuring the body fat using the electronic device, and a health management system using the electronic device and method, wherein the electronic device for measuring body fat includes an electronic appliance, two pairs of electrodes provided on the electronic appliance, the two pairs of electrodes being able to be contacted by a subject, and a measuring portion for measuring a bio-impedance of the subject and calculating body fat data of the subject using the measured bio-impedance when the subject contacts the two pairs of electrodes. The health management system includes an Internet server for comparing data of the subject to a standardized set of data and outputting health information based on the comparison result to the subject.

### BACKGROUND OF THE INVENTION

1. Field of the Invention

The present invention relates to an electronic device for measuring body fat, a method of measuring body fat using the same, and a health management system using the device and method. More particularly, the present invention relates to an electronic device for measuring body fat that may be incorporated into an electronic appliance, and a method of measuring body fat using the device and a health management system using the device and the method.

2. Description of the Related Art

A human body is comprised of four principal components: body fluid, protein, fat, and an inorganic substance. The body fluid, protein, fat, and inorganic substance are present in a ratio of roughly 55:20:20:5, respectively, in the weight of an average human body, although the ratio varies with sex or age of an individual. The quantities of the four principal components can be determined from the total body water (TBW) since protein and TBW, the main components of muscle, are interactively proportional to each other. In other words, sound muscle comprises about 73% by weight of water and about 27% by weight of protein. The inorganic substance is a component that chiefly forms bone, and the weight of the bone is closely associated with the muscle quantity. That is, the quantities of the protein and inorganic substance can be obtained from the TBW, and thus, body fat can be obtained by subtracting the quantities of the TBW, protein, and inorganic substance from the total weight of a human body. In recent years, bioelectrical impedance analysis (BIA) has been considered one of the most precise and accessible body fat measuring methods. Other methods such as hydrodensitometry, computerized tomography (CT) assessment of fat, and measurement of skinfold thickness and circumference have also been used to measure body fat.

In the BIA method, body fat is measured on the basis of an inversely proportional relationship between the TBW and bioelectric impedance. The BIA method has advantages of being simple and providing rapid non-invasive measurements. When a weak alternating current (AC) electric signal is applied to a human body, electricity flows through the TBW, which has high conductivity. The width of a path through which electricity flows is determined by an amount of to the TBW, and bio-impedance is obtained by measuring the electrical signal, which flows out from the path.

A principle of calculating the quantities of body components from bio-impedance is as follows. At the outset, a weak AC current of about 1 mA in a frequency band of 50 kHz is applied to a human body. While the current is flowing through the human body, bio-impedance is measured, and the TBW is obtained using the bio-impedance. Thus, the quantities of protein and inorganic substance are obtained from the TBW. The amount of body fat is then calculated by subtracting the quantities of protein, inorganic substance, and the TBW from the total weight of the human body.

To more effectively manage health, body fat should be measured using the foregoing BIA method in an easier and more accessible manner.

### SUMMARY OF THE INVENTION

The present invention is therefore directed to an electronic device for measuring body fat, a method of measuring body fat using the same, and a health management system using the device and method, which substantially overcome one or more of the problems due to the limitations and disadvantages of the related art.

It is a feature of an embodiment of the present invention to provide an electronic device for measuring body fat, which is easily accessible in a home and includes electrodes for measuring bio-impedance to easily measure body fat.

It is another feature of an embodiment of the present invention to provide a method of measuring body fat using the device.

It is still another feature of an embodiment of the present invention to provide a health management system, which measures body fat using an electronic device accessible in a home and facilitates management of the health of a subject based on the measured result.

At least one of the above features and other advantages may be provided by an electronic device for measuring body fat including an electronic device for measuring body fat including an electronic appliance, two pairs of electrodes provided on the electronic appliance, the two pairs of electrodes being able to be contacted by a subject, and a measuring portion for measuring a bio-impedance of the subject and calculating body fat data of the subject using the measured bio-impedance when the subject contacts the two pairs of electrodes.

The measuring portion may include a circuit portion for applying a current to the subject via one of the two pair of electrodes and for measuring a voltage taken at the subject via the other of the two pairs of electrodes when the subject is in contact with the two pairs of electrodes, an input portion for receiving personal information including at least a height and weight of the subject, an information processing portion for calculating the bio-impedance using the measured current and voltage and calculating the body fat data using the personal information, and an output portion for outputting the calculated body fat data.

The information processing portion may include an analog/digital converter for converting the current and voltage to digital values and a controller for calculating the bio-impedance using the digitally converted current and voltage and calculating the body fat data using the bio-impedance and the personal information of the subject.

The electronic device may further include a storing portion for storing the personal information and body fat data of the subject.

The information processing portion may further include a communicating portion connected to an Internet server for communicating information regarding the calculated body fat data, and wherein the controller provides the personal information and the body fat data of the subject to the Internet server, receives corresponding health information from the Internet server, and communicates the health information to the subject via the output portion.

At least one of the above features and other advantages may be provided by a method of measuring body fat of a subject by measuring a bio-impedance of the subject that maintains contact with two pairs of electrodes installed in positions of an electronic appliance, which electrodes the subject contacts to use the electronic appliance, the method including receiving personal information regarding the subject when the subject maintains contact with the two pairs of electrodes for more than a predetermined amount of time, measuring the bio-impedance of the subject, and calculating body fat data of the subject using the received personal information and the measured bio-impedance and informing the subject of the calculated body fat data.

The method may further include outputting a question about whether the subject desires to take a body fat measurement and receiving an answer to the question from the subject, if the subject maintains contact with the two pairs of electrodes for more than the predetermined amount of time.

In the method, receiving the personal information of the subject may include outputting a subject list stored in the electronic device, receiving an item of the subject list selected by the subject, and reading information corresponding to the received item among information stored in the electronic device. Alternatively, receiving the personal information of the subject may include receiving information including at least a weight and height of the subject.

The method may further include connecting with an Internet server and transmitting subject information including at least the body fat data to the Internet server, receiving health information corresponding to the body fat data of the subject from the Internet server, and outputting the health information.

At least one of the above features and other advantages may be provided by a health management system including an electronic device, capable of being connected to the Internet, for measuring a body fat of a subject by measuring a bio-impedance of the subject when the subject maintains contact with two pairs of electrodes disposed in predetermined positions of an electronic appliance, which electrodes are contacted by the subject to use the electronic appliance, a database storing identification data of a plurality of subjects and corresponding personal information including at least a weight and height and health information relative to body fat data corresponding to each of the plurality of subjects, and an Internet server for conducting a comparison of personal information of a subject including body fat data of the subject against a set of standardized data to produce a comparison result and outputting health information based on the comparison result to the electronic device.

In the health management system, the electronic device for measuring the body fat may include an electronic appliance, two pairs of electrodes being installed in predetermined positions of the electronic appliance, which electrodes the subject contacts to use the electronic appliance, a circuit portion for applying a current to the subject through one pair of the two pairs of electrodes and measuring a voltage taken at the subject through the other pair of the two pairs of electrodes, an input portion for receiving personal information including at least a weight and height of the subject, an information processing portion for calculating bio-impedance using the measured current and voltage and calculating the body fat data using the received personal information of the subject, an output portion for outputting the calculated body fat data, and a communicating portion for providing communication between the information processing portion and the Internet server, the communicating portion exchanging information with the Internet server.

In the device or the system as described above, the two pairs of electrodes may be disposed on one or more handles of the electronic appliance. Alternatively, the two pairs of electrodes may be disposed on buttons on the electronic appliance.

The information processing portion may include an analog/digital converter for converting the current and voltage measured in the circuit portion to digital values and a controller for calculating the bio-impedance using the digitally converted current and voltage and for calculating body fat data using the bio-impedance and the personal information of the subject.

The system may further include a storing portion for storing the personal information and body fat data of the subject.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a block diagram of an electronic device for measuring body fat according to an embodiment of the present invention;

FIG. 2 illustrates a subject measuring body fat using an electronic device according to an embodiment of the present invention, in which two pairs of electrodes are provided on a handle of an electronic appliance, e.g., a refrigerator;

FIG. 3 illustrates a subject measuring body fat using an electronic device according to another embodiment of the present invention, in which two pairs of electrodes are provided on buttons of an electronic appliance, e.g., a microwave oven; and

FIG. 4 is a flowchart illustrating a method of measuring body fat according to an embodiment of the present invention.

### DETAILED DESCRIPTION OF THE INVENTION

Korean Patent Application No. 2003-69703, filed on Oct. 7, 2003, in the Korean Intellectual Property Office, and entitled: “Electronic Device for Measuring Body Fat, Method of Measuring Body Fat Using the Same, and Health Management System Using the Device and Method,” is incorporated by reference herein in its entirety.

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals and characters indicate like elements throughout. In the context of the present invention, the term electronic appliance may represent any electronic device and is not intended to be limited to the exemplary embodiments described herein.

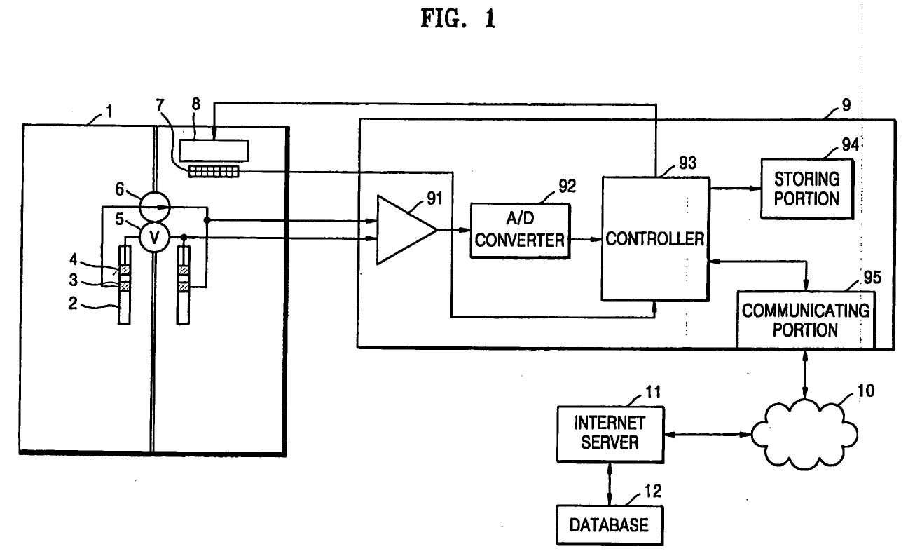

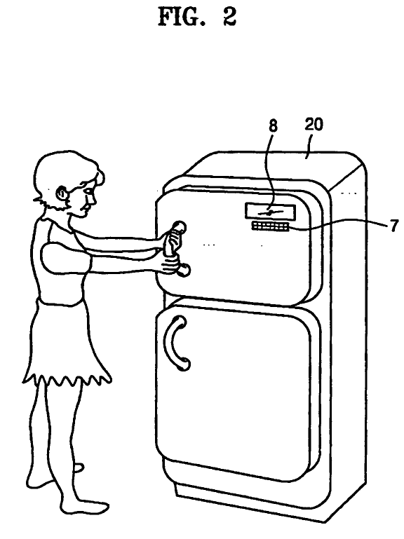

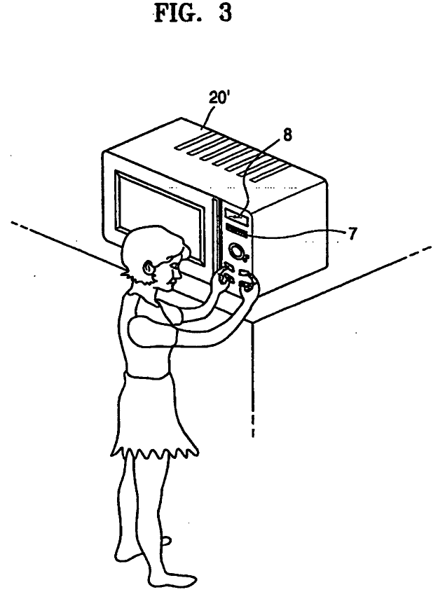

FIG. 1 is a block diagram of an electronic device 1 for measuring body fat according to an embodiment of the present invention. Referring to FIG. 1, the electronic device 1 includes two contacts 2 on a front side of an electronic appliance, e.g., 20 of FIG. 2 or 20′ of FIG. 3. Each of the contacts 2 may contact one of a subject's hands. The contacts 2 include a first pair of electrodes 3 and a second pair of electrodes 4. The contacts 2 are spaced a predetermined distance apart from each other. The first pair of electrodes 3 is connected to a current generator 6. The second pair of electrodes 4 is connected to a voltmeter 5.

The electronic device 1 further includes an input portion 7, e.g., a keyboard or keypad, for inputting data and an output portion 8, e.g., a display, for communicating data to the subject. The electronic device 1 further includes an information processing portion 9, which is connected to the current generator 6 and voltmeter 5, embedded in the electronic device 1.

The contacts 2 are not necessarily designed to be gripped by both hands. For example, although the contacts 2 may be provided on one or more handles of the electronic appliance, the contacts 2 may be alternatively or additionally provided on buttons on the electronic appliance, on which two pairs of electrodes 3 and 4 are disposed. In the case of the one or more handles, bio-impedance may be measured when the handle is manually gripped. In the case of the buttons, bio-impedance may be measured when the buttons are manually pressed.

FIGS. 2 and 3 illustrate exemplary embodiments of an electronic device for measuring body fat according to an embodiment of the present invention including two pairs of electrodes installed on a front side of an electronic appliance, e.g., a kitchen appliance. Specifically, FIG. 2 illustrates a subject measuring body fat using an electronic device according to an embodiment of the present invention, in which two pairs of electrodes are provided, e.g., linearly, on a handle of an electronic appliance 20, i.e., a refrigerator. FIG. 3 illustrates a subject measuring body fat using an electronic device according to an embodiment of the present invention, in which two pairs of electrodes are provided on buttons of an electronic appliance, i.e., a microwave oven 20′.

When a subject contacts each pair of electrodes 3 and 4 with each hand, the subject's body functions as an impedance, thereby forming a closed circuit. As a result, a current of about 1 mA generated from the current generator 6 of FIG. 1 is transmitted through the first pair of electrodes 3 and into the subject's body via the hands. A voltage taken at the bio-impedance of the body is measured using the voltmeter 5 through the second pair of electrodes 4.

The input portion 7 allows the subject to input data, and the output portion 8 displays data and information to the subject. Although the output portion 8 usually communicates data specific to a particular electronic appliance, for example, the present internal temperature of a refrigerator or time remaining in a cooking operation of a microwave oven, the output portion 8 may also display information on health or body components, like body fat, during a measurement of body fat.

Although the output portion 8 is exemplarily described as being a display unit, the output portion 8 may further include a speaker or a light-emitting diode (LED) to communicate data to the subject.

The information processing portion 9 includes an amplifier 91, an analog/digital (A/D) converter 92, a controller 93, a storing portion 94, and a communicating portion 95. In this construction, the electronic device 1 is able to interactively acquire data from the subject via the input portion 7 to measure body fat, and display data to the subject via the output portion 8.

The amplifier 91, which is connected to the current generator 6 and the voltmeter 5, receives a current applied to the body and a voltage generated in the body, and amplifies the current and voltage. The A/D converter 92 converts the amplified current and voltage to digital values. The controller 93 calculates the bio-impedance using the converted current and voltage, and thus, calculates body fat using the bio-impedance.

By knowing the subject's weight (Wt) and height (Ht), and by measuring the subject's bio-impedance (R), total body water (TBW) can be obtained from the following regression analysis equation (1):
TBW=a*Ht²/R+b*Wt+c*age+d (1).

Here, a, b, c, and d are weight constants. In equation 1, Ht and Wt are requisite parameters, and age (or sex) is an optional parameter.

Fat-free mass (FFM), i.e., the mass of a portion of body which is not fat, includes about 73% by weight of TBW, about 20% by weight of protein, about 6% by weight of inorganic substance, and about 1% by weight of ash. The FFM can be expressed using the TBW shown in the equation (2):
FFM=TBW/0.73 (2).

Thus, body fat weight is obtained by subtracting FFM from Wt. Percent body fat (% BF) can be expressed using the Wt and body fat weight as shown in the following equation (3):
% BF=(fat/Wt)*100 (3)

The controller 93 stores body fat data, such as the calculated body fat or percent body fat (% BF), along with personal information of the subject, for example, name, height, weight, age, and sex, in the storing portion 94. The storing portion 94 stores the personal information input by the subject during an initial body fat examination, updates the body fat data, such as body fat or percent body fat (% BF), whenever the subject measures body fat, and may provide a variation of body fat to the subject in response to a request by the subject. In addition to information input by the subject, the storing portion 94 may store preset information on appropriate body fat according to age, sex, height, or weight.

The controller 93 also receives personal information or necessary information regarding the subject via the input portion 7 and communicates information to the subject via the output portion 8. The information communicated to the subject may be information on health, which is received through the communicating portion 95 from an Internet server 11 via the Internet 10.

The Internet server 11 further includes a database 12, which stores information on the subject's health, for example, recommendable foods, caloric intake, and workouts according to the subject's body fat.

The database 12 may further store identifications (IDs) of multiple subjects and personal information of a subject having a specific ID, such as height, weight, age, sex, or present body fat data.

The Internet server 11 may receive only the ID of a subject and body fat data or all of the subject's personal information and body fat data from the controller 93.

The Internet server 11 searches optimum health information in the database 12 based on information provided from the electronic device 1, such as the height, weight, age, sex, or current body fat data of the subject, and provides the information to the controller 93.

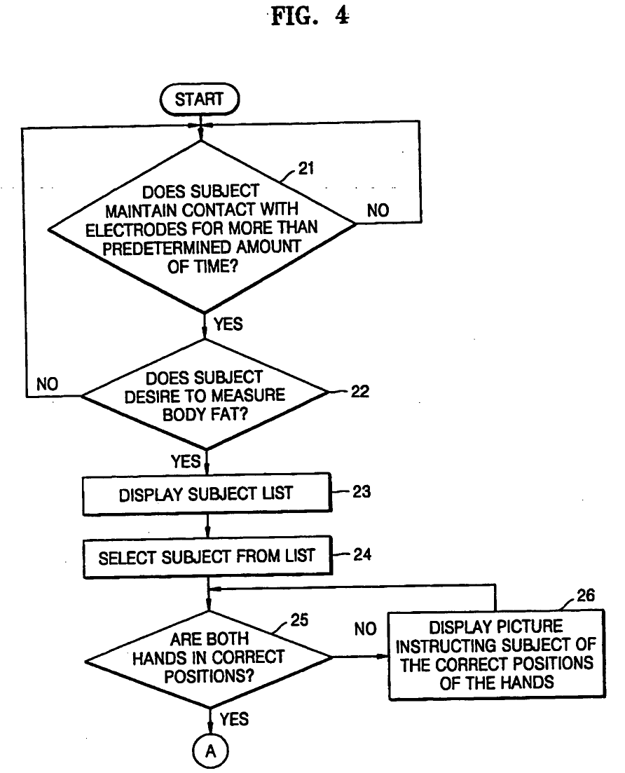

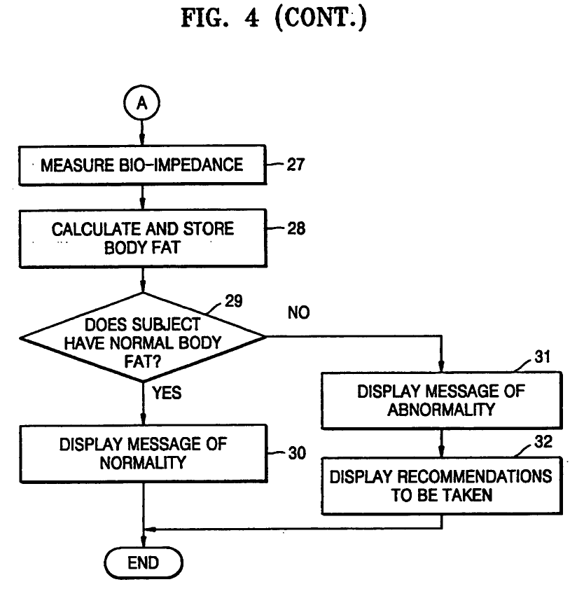

A process of measuring body fat according to an embodiment of the present invention, which is controlled by the controller 93, will now be described with reference to FIG. 4. In operation 21, the controller 93 monitors the contact of a subject with the first and second pairs of electrodes 3 and 4 for a predetermined amount of time. If it is determined that the subject has maintained contact with the electrodes 3 and 4 for more than the predetermined amount of time, in operation 22, the controller 93 queries the intention of the subject. Specifically, the controller 93 uses the output portion 8 to ask the subject if he/she desires to measure his/her body fat. The subject uses the input portion 7 to provide an answer (e.g., yes or no) indicating whether the subject intentionally contacted the electrodes 3 and 4 to take a body fat measurement.

In operation 23, the controller 93 displays a subject list stored in the storing portion 94, and the subject selects himself/herself from the list. Alternatively, if the subject is not presently included in the subject list, the subject may input his/her own personal information.

In operation 25, the controller 93 monitors whether both hands of the subject are in correct positions, i.e., positions that allow the two pairs of electrodes 3 and 4 to appropriately measure bio-impedance. For example, to monitor whether both hands are in correct positions, it may be determined whether a voltage measured using the current generator 6 and the voltmeter 5 is greater than a predetermined value.

In operation 26, if one or both hands are in incorrect positions, a picture instructing the subject of the correct positions of the hands is displayed. In operation 27, if both hands are in the correct positions, bio-impedance is measured. In operation 28, body fat or percent body fat is calculated from the measured bio-impedance and stored for the corresponding subject in the storing portion 94.

In operation 29, it is determined whether the measured body fat is appropriate relative to the weight, height, and/or age of the subject by comparing the personal information of the subject, including body fat data of the subject against a set of standardized data. If the body fat is appropriate, in operation 30, a message of normality is communicated to the subject. If the body fat is inappropriate, in operation 31, a message of abnormality, namely, an indication of too much or too little body fat, is communicated to the subject. Further, in operation 32, after receiving the message of abnormality, recommendations of an appropriate remedy may be communicated to the subject. The remedy recommendations may be provided from the Internet server 11 or preset in the storing portion 94 when the electronic device is produced. Here, the message of normality or abnormality may be audibly provided through a speaker of the output portion 8.

According to the present invention, because an electronic device for measuring fat according to an embodiment of the present invention can be incorporated into conventional household appliances, which are usually always turned on and readily accessible, body fat can be easily measured without the need of an additional fat measuring device.

Moreover, the electronic device for measuring fat according to an embodiment of the present invention may be incorporated into handles or buttons of household appliances for storing or cooking foods, such as a refrigerator or a microwave oven. Thus, with reference to measurement results, a subject can instantaneously control caloric intake and manage weight more effectively.

Further, the present invention can inform the subject of useful information on health in accordance with the measurement results by accessing an Internet server via the Internet, thus facilitating access to information.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. An electronic device for measuring body fat, comprising:
an electronic appliance;
two pairs of electrodes provided on the electronic appliance, the two pairs of electrodes being able to be contacted by a subject; and
a measuring portion for measuring a bio-impedance of the subject and calculating body fat data of the subject using the measured bio-impedance when the subject contacts the two pairs of electrodes.

2. The electronic device as claimed in claim 1, wherein the two pairs of electrodes are disposed on one or more handles of the electronic appliance.

3. The electronic device as claimed in claim 1, wherein the two pairs of electrodes are disposed on buttons on the electronic appliance.

4. The electronic device as claimed in claim 1, wherein the measuring portion comprises:
a circuit portion for applying a current to the subject via one of the two pair of electrodes and for measuring a voltage taken at the subject via the other of the two pairs of electrodes when the subject is in contact with the two pairs of electrodes;
an input portion for receiving personal information including at least a height and weight of the subject;
an information processing portion for calculating the bio-impedance using the measured current and voltage and calculating the body fat data using the personal information; and
an output portion for outputting the calculated body fat data.

5. The electronic device as claimed in claim 4, wherein the information processing portion comprises:
an analog/digital converter for converting the current and voltage to digital values; and
a controller for calculating the bio-impedance using the digitally converted current and voltage and calculating the body fat data using the bio-impedance and the personal information of the subject.

6. The electronic device as claimed in claim 5, further comprising a storing portion for storing the personal information and body fat data of the subject.

7. The electronic device as claimed in claim 5, wherein the information processing portion further comprises a communicating portion connected to an Internet server for communicating information regarding the calculated body fat data,
and wherein the controller provides the personal information and the body fat data of the subject to the Internet server, receives corresponding health information from the Internet server, and communicates the health information to the subject via the output portion.

8. A method of measuring body fat of a subject by measuring a bio-impedance of the subject that maintains contact with two pairs of electrodes installed in positions of an electronic appliance, which electrodes the subject contacts to use the electronic appliance, the method comprising:
receiving personal information regarding the subject when the subject maintains contact with the two pairs of electrodes for more than a predetermined amount of time;
measuring the bio-impedance of the subject; and
calculating body fat data of the subject using the received personal information and the measured bio-impedance and informing the subject of the calculated body fat data.

9. The method as claimed in claim 8, further comprising outputting a question about whether the subject desires to take a body fat measurement and receiving an answer to the question from the subject, if the subject maintains contact with the two pairs of electrodes for more than the predetermined amount of time.

10. The method as claimed in claim 8, wherein receiving the personal information of the subject comprises:
outputting a subject list stored in the electronic device;
receiving an item of the subject list selected by the subject; and
reading information corresponding to the received item among information stored in the electronic device.

11. The method as claimed in claim 8, wherein receiving the personal information of the subject comprises receiving information including at least a weight and height of the subject.

12. The method as claimed in claim 8, further comprising:
connecting with an Internet server and transmitting subject information including at least the body fat data to the Internet server;
receiving health information corresponding to the body fat data of the subject from the Internet server; and
outputting the health information.

13. A health management system, comprising:
an electronic device, capable of being connected to the Internet, for measuring a body fat of a subject by measuring a bio-impedance of the subject when the subject maintains contact with two pairs of electrodes disposed in predetermined positions of an electronic appliance, which electrodes are contacted by the subject to use the electronic appliance;
a database storing identification data of a plurality of subjects and corresponding personal information including at least a weight and height and health information relative to body fat data corresponding to each of the plurality of subjects; and
an Internet server for conducting a comparison of personal information of a subject including body fat data of the subject against a set of standardized data to produce a comparison result and outputting health information based on the comparison result to the electronic device.

14. The system as claimed in claim 13, wherein the electronic device for measuring the body fat comprises:
an electronic appliance;
two pairs of electrodes being installed in predetermined positions of the electronic appliance, which electrodes the subject contacts to use the electronic appliance;
a circuit portion for applying a current to the subject through one pair of the two pairs of electrodes and measuring a voltage taken at the subject through the other pair of the two pairs of electrodes;
an input portion for receiving personal information including at least a weight and height of the subject;
an information processing portion for calculating bio-impedance using the measured current and voltage and calculating the body fat data using the received personal information of the subject;
an output portion for outputting the calculated body fat data; and
a communicating portion for providing communication between the information processing portion and the Internet server, the communicating portion exchanging information with the Internet server.

15. The system as claimed in claim 14, wherein the two pairs of electrodes are disposed on one or more handles of the electronic appliance.

16. The system as claimed in claim 14, wherein the two pairs of electrodes are disposed on buttons on the electronic appliance.

17. The system as claimed in claim 14, wherein the information processing portion comprises:
an analog/digital converter for converting the current and voltage measured in the circuit portion to digital values; and
a controller for calculating the bio-impedance using the digitally converted current and voltage and for calculating body fat data using the bio-impedance and the personal information of the subject.

18. The system as claimed in claim 17, further comprising a storing portion for storing the personal information and body fat data of the subject.
